(19) United States
(12) Patent Application Publication
(10) Pub. No.: US 2024/0148636 A1
(43) Pub. Date: May 9, 2024

(54) Sedurre Skincare Formulations

(71) Applicant: Y Terrichele Bradley, Plano, TX (US)

(72) Inventor: Y Terrichele Bradley, Plano, TX (US)

(21) Appl. No.: 17/983,809

(22) Filed: Nov. 9, 2022

(51) Int. Cl. A61K 8/92; A61K 8/34; A61K 8/67; A61K 8/73; A61K 8/9789; A61K 8/9794; A61Q 5/12; A61Q 19/00

(52) U.S. Cl. CPC A61K 8/922; A61K 8/345; A61K 8/678; A61K 8/73; A61K 8/927; A61K 8/9789; A61K 8/9794; A61Q 5/12; A61Q 19/007

(57) ABSTRACT

The present invention provides topical skin and hair care formulas comprised of natural ingredients such as butters and oils that softens, moisturizes and hydrates skin and hair including facial hair like beards. Furthermore, the invention includes palmitic, linoleic, stearic and oleic fatty acids, ingredients that balance oils on the skin. The invention promotes circulation and healthy skin cell growth with antioxidants vitamins like vitamins A, E and F. Triglycerides help nourish and condition the skin. In addition, cetyl esters not only condition the skin, but help lock in moisture.

### FIELD OF INVENTION

The field of the invention relates to topically applied cosmetic products that moisturize skin and hair.

The body butter and body oil can be applied all over the body, especially rough dry skin, fine lines for mature skin and areas of itchy skin due to eczema. The body butter and body oil can be applied to hair also. It helps minimize irritation caused by environmental factors such as dry weather as well as inflammatory skin conditions. It includes ingredients that include vitamins A and E which promote antioxidants, protecting the skin cells from premature aging and dull-looking skin. The beard oil, beard balm and beard moisturizer are rich in omega-3, omega-6 and omega-9 and filled fatty acids which maximizes hair growth. This special formulated product reduces breakage, split ends, and may help provide thicker and fuller beards. It also gets rid of dandruff and helps tame unruly beards as it softens and changes the texture of the hair while creating styles with ease.

### BACKGROUND OF INVENTION

While many products are sold to reduce rough dry skin and stimulate and soften beard growth none are specifically provided that reduce rough dry skin by improving the texture of the skin and its elasticity and hydration. The body butter not only reduces rough dry skin by improving the texture of the skin and its elasticity and hydration, but it relieves itchy symptoms due to eczema. It also reduces fine lines for mature skin. Products to improve beard growth and texture such as softness are also lacking. It is the purpose of the present invention to provide these improvements.

### BRIEF SUMMARY OF THE INVENTION

The present invention provides topically applied skin care products that reduce rough dry skin, by improving skin's texture, elasticity and hydration and moisturizes skin. These skin care products are safe to apply to the face and neck area and may be applied to other parts of the body. It is another object of this invention to provide moisturizing skin and hair care products that are easy to use, relatively simple to manufacture, and are cost effective.

In one embodiment of the invention, the formulas of the present invention stimulate hair and beard growth, reduce dandruff and relieve itchy scalp. The products moisturize the skin underneath and softens the beard.

The term “emulsifier” refers to a substance capable of forming or promoting an emulsion. An emulsion is a mixture of two or more liquids that are normally immiscible (nonmixable or unblendable). Emulsions are part of a more general two-phase used systems of matter called colloids. Although the terms colloid and emulsions are sometimes interchangeable, emulsions should be used when both the dispersed and the continuous phase are liquids. In an emulsion, one liquid (the dispersed phase) is dispersed in the other (the continuous phase). The beard moisturizing formula of the invention is an emulsion, a blend of water and oil brought together by using an emulsifier such as Olivem 1000 and manufactured using palm oil or other emulsifiers which creates a homogenous blend such as, for example, a lotion.

In the beard moisturizing formula of the invention a “preservative” is added to protect against bacteria, fungus and yeast. The term “preservative” can be a naturally occurring or synthetically created chemical that is put in cosmetic products to stop spread of bacteria and the decomposition of the formula through growth of microbial or unwanted chemical modifications. One example of such a preservative is Leucidal SF Complete. While a preservative may be added to any of the embodiments of the present invention, it is only required in the beard moisturizing composition.

The term “fragrances” refers to a combination of chemicals that gives each product its distinct scent. Chemical ingredients may be derived from synthetic or natural raw materials that can add or mask a scent. The ingredients are capable of imparting or modifying the odor of skin or hair.

Fragrance, ranging from 0% to 3%, may be selected from a group comprised of essential oils, synthetically compounded from aroma chemicals or all-natural fragrance without the use of synthetics. In a preferred embodiment, the fragrance is a combination of sandalwood and vanilla or coconut milk, milky sandalwood and hawthorne.

### DETAILED DESCRIPTION OF THE PREFERRED EMBODIMENTS

Body Butter

In one embodiment the invention provides a body butter that reduces rough dry skin, by improving skin's texture, elasticity and hydration and moistures skin. The body butter absorbs quickly and reduces dryness by locking in moisture. Also, it reduces fine lines in mature skin. The body butter is comprised essentially of oils and butters selected from Shea Butter, Mango Butter, Fractionated Coconut Oil, Apricot Oil, Avocado Oil, Beeswax, Arrowroot Powder, Vitamin E, and Fragrance.

The butters and beeswax are heated and held at 170° F. for at least 20 minutes. The oils are added to the butter and beeswax mixture while stirring gently. The mixture of the oil, butter and beeswax is allowed to gently cool and Vitamin E is added. The arrowroot powder is then poured into the mixture. The mixture is then placed in a cooled environment, for instance, in the refrigerator or freezer to cool down and slightly solidify, but are not allowed to harden completely. The mixture is mixed on medium to high speed for about 20 to 30 minutes until it is fluffy using a hand mixer. The fragrance is then added to the fluffy body butter and mixed until it is completely scented.

The body butter is gently applied to the skin and rubbed into skin as it absorbs quickly into the skin.

In one embodiment, the body butter is comprised of from about 20% to about 45% by weight Shea Butter, from about 20% to about 30% by weight Mango Butter, from about 20% to about 30% by weight Fractionated Coconut Oil, from about 5% to about 20% by weight Apricot Oil, from about 5% to about 15% by weight Avocado Oil, from about 5% to about 15% by weight Beeswax, from about 2% to about 10% by weight Arrowroot Powder, from about 1% to about 2% by weight Vitamin E and from about 1% to about 3% by weight Fragrance.

In a most preferred embodiment, the body butter is comprised of a 30% Shea Butter, 22% Mango Butter, 22% Fractionated Coconut Oil, 10% Apricot Oil, 5% Avocado Oil, 5% Beeswax 4% Arrowroot Powder, 1% Vitamin E and 1% Fragrance.

The butters and beeswax are heated and held at 170° F. for at least 20 minutes. The oils are added to the butter and beeswax mixture while stirring gently. The mixture of the oil, butter and beeswax is allowed to gently cool and the Vitamin E is added. The arrowroot powder is then poured into the mixture. The mixture is then placed in a below 40° F. environment to cool down and slightly solidify, but is not allowed to harden completely. Take the mixture out of the refrigerator and with a hand mixer, mix on medium to high speed for about 20 to 30 minutes until it is fluffy. ¶

Body Oil

Another embodiment is the topical cosmetic product of the invention provides a body oil that absorbs quickly on the skin promoting hydration and moisturizing the skin thereby improving texture and elasticity. The body oil treats rough dry skin while sealing in moisture. It also reduces fine lines in all types of skin. The body oil is comprised of Apricot Oil, Fractionated Coconut Oil, Avocado Oil, Vitamin E, and fragrance.

In one embodiment, the body oil is comprised of from about 30% to about 50% by weight Apricot Oil, from about 20% to about 40% by weight Fractionated Coconut Oil, from about 20% to about 30% by weight Avocado Oil, from about 1% to about 2% by weight Vitamin E, and from about 1% to about 3% by weight fragrance.

In the most preferred embodiment, the body oil is comprised of 45% Apricot Oil, 30% Fractionated Coconut Oil, 22.5% Avocado Oil, 0.5% Vitamin E, and 1% fragrance.

The body oil is prepared by mixing the Apricot Oil, Fractionated Coconut Oil and Avocado Oil together. Then, adding the Vitamin E to the oil mixture. Last, add the fragrance to the mixture, and stir gently.

Use the body oil to reveal smooth and glowing skin. Smooth a small amount on damp skin after showering or bathing to seal in moisture. Let body air dry or pat gently with a towel.

Beard Oil

In another embodiment the topical cosmetic product of the invention provides a skincare beard oil that stimulates hair growth, reduces dandruff and relieves itching, while softening the beard and moisturizing the skin. The beard oil of the present embodiment of the invention conditions the beard to provide a healthy, smooth soft appearance with a nice shine.

The beard oil of the present embodiment is comprised of Sweet Almond Oil, Apricot Oil, Jojoba Oil, Argan Oil, Hemp Seed Oil, Vitamin E and Fragrance.

One embodiment of the beard oil of the present invention is comprised by weight ranges from 20% to 35% Sweet Almond Oil, 20% to 35% Apricot Oil, 20% to 35% Jojoba Oil, 20% to 35% Argan Oil, 5% to 15% Hemp Seed Oil, 1% to 2% Vitamin E and 1% to 3% Fragrance.

The most preferred embodiment of the beard oil of the present invention comprises 26.5% by weight Sweet Almond Oil, 20% by weight Apricot Oil, 20% by weight Jojoba Oil, 20% by weight Argan Oil, 10% by weight Hemp Seed Oil, 1.5% by weight Vitamin E and 2% by weight Fragrance.

Use the beard oil on clean skin. The beard is dried with a towel so it is damp with water, but not wet. Apply the beard oil on the damp beard and then comb or brush the beard.

Beard Balm

Another embodiment of the invention provides a beard balm. The beard balm stimulates hair growth, reduces dandruff and relieves itching, while softening the beard and the skin.

The beard balm is comprised of Sweet Almond Oil, Apricot Oil, Jojoba Oil, Argan Oil, Hemp Seed Oil, Beeswax, Shea Butter, Mango Butter, Vitamin E, and Fragrance.

One embodiment of the beard balm of the present invention is comprised by weight ranges from about 20% to about 35% Sweet Almond Oil, from about 10% to about 20% Apricot Oil, from about 5% to about 15% Jojoba Oil, from about 5% to about 15% Hemp Seed Oil, from about 5% to about 20% Beeswax, from about 5% to about 20% Shea Butter, from about 5% to about 20% Mango Butter, from about 1% to about 2% Vitamin E and from about 1% to about 3% Fragrance.

A preferred embodiment of the beard balm of the present invention is comprised of 20.5% Sweet Almond Oil, 10% Apricot Oil, 10% Jojoba Oil, 10% Argan Oil, 10% Hemp Seed Oil, 12% Beeswax, 12% Shea Butter, 12% Mango Butter, 1.5% Vitamin E, and 2% Fragrance.

The butters and beeswax are heated and held at 170° F. for at least 20 minutes. The oils are added to the butters and beeswax mixture while gently stirring. The mixture is allowed to cool but is not allowed to harden. Vitamin E is added to the mixture. The mixture is placed in a cooling device to cool down and solidify.

Use the beard balm on clean skin. Dry the bread with a towel so it is damp with water, but not wet. Apply the beard balm on the damp beard and then comb or brush the beard to maintain, shape and style.

Beard Moisturizing Formula

In another embodiment the invention provides a Beard Moisturizer Formula comprising Sweet Almond Oil, Apricot Oil, Jojoba Oil, Hemp Seed Oil, Shea Butter, Mango Butter, Emulsifier, Cetyl Alcohol, Beeswax, Distilled Water, Glycerin, Xanthan Gum, Oat Extract, and Bamboo Extract, Vitamin E, Fragrance and Preservative.

One embodiment of the beard moisturizing formula of the invention comprises from about 2% to about 10% by weight Sweet Almond Oil, from about 2% to about 10% by weight Apricot Oil, from about 2% to about 10% by weight Jojoba Oil, from about 2% to about 10% by weight Argan Oil, from about 1% to about 5% by weight Hemp Seed Oil, from about 1% to about 5% by weight Shea Butter, from about 1% to about 5% by weight Mango Butter, from about 1% to about 10% by weight Emulsifier, from about 2% to about 10% by weight Beeswax, from about 45% to about 60% Distilled Water, from about 2% to about 10% by weight Glycerin, from about 0.2% to about 3% by weight Xanthan Gum, from about 5% to about 15% by weight Oat Extract, from about 5% to about 15% by weight Bamboo Extract, from about 1% to about 2% by weight Vitamin E, from about 1% to about 3% by weight Fragrance, and from about 1% to about 5% Preservative.

In a preferred embodiment the invention provides a Beard Moisturizer Formula comprising 3% Sweet Almond Oil, 3% Apricot Oil, 3% Jojoba Oil, 2% Hemp Seed Oil, 2.5% Shea Butter, 2.5% Mango Butter, 5% Emulsifier, 3% Cetyl Alcohol, 2% Beeswax, 51.5% Distilled Water, 3% Glycerin, 0.5% Xanthan Gum, 5% Oat Extract, 5% Bamboo Extract, 1% Vitamin E, 2% Fragrance and 3% Preservative.

### Example 1

Body Butter Formula:

The body butter is prepared by combining Shea butter, mango butter, beeswax, fractionated coconut oil, apricot oil and avocado oil together. Then, heat the mixture until it is melted. The mixture is gently stirred while heating for at least 20 minutes. Remove the mixture from the heat. Let sit for 5 to 10 minutes. Then, add the Vitamin E and stir gently. Add the arrowroot powder to the mixture and gently stir. Place the mixture in cooling device at least 40° F. The mixture is allowed to cool, and slightly solidify. With a hand mixer or standalone mixer, mix the slightly hardened body butter for about 20 to 30 minutes until fluffy and creamy. Then, add the fragrance to the fluffy and creamy body butter thoroughly.

In the most preferred embodiment, the body butter consists of 30% Shea Butter, 22% Mango Butter, 22% Fractionated Coconut Oil, 10% Apricot Oil, 5% Avocado Oil, 5% Beeswax, 4% Arrowroot Powder, 1% Vitamin E, and 1% Fragrance.

### Example 2: Body Oil Formula

The body oil of the present invention is prepared by combining and thoroughly mixing the Apricot Oil, Fractionated Coconut Oil, Avocado Oil, Vitamin E, and Fragrance at room temperature.

One embodiment of the body oil of the present invention is comprised of from about 30% to about 50% by weight Apricot Oil, from about 20% to about 40% by weight Fractionated Coconut Oil, from about 20% to about 30% by weight Avocado Oil, from about 1% to about 2% by weight Vitamin E, and from about 1% to about 3% by weight Fragrance.

The most preferred embodiment of the body oil consists essentially of 45% Apricot Oil, 30% Fractionated Coconut Oil, 22.5% Avocado Oil, 1.5% Vitamin E, and 1% Fragrance.

The body oil can be applied to the body by rubbing with your hands and provides relief of rough dry skin, reduces fine lines from mature skin, relieves itchy skin which are symptoms of eczema.

### Example 3: Beard Oil Formula

The beard oil of the present invention is prepared by combining and thoroughly mixing the Sweet Almond Oil, Apricot Oil, Jojoba Oil, Argan Oil, Hemp Seed Oil, Vitamin E and Fragrance.

In the most preferred embodiment the Beard oil consists of 26.5% Sweet Almond Oil, 20% Apricot Oil, 20% Jojoba Oil, 20% Argan Oil, 10% Hemp Seed Oil, 1.5% Vitamin E and 2% Fragrance.

The beard oil can be applied to the beard and hair.

### Example 4: Beard Balm Formula

A beard balm of the present invention is comprised of Sweet Almond Oil, Apricot Oil, Jojoba Oil, Argan Oil, Hemp Seed Oil, Beeswax, Shea Butter, Mango Butter, Vitamin E and Fragrance.

The beard balm is prepared by mixing shea butter, mango butter and beeswax together and heat for at least 20 minutes to a temperature of 170° F. while gently stirring, Then, add Sweet Almond Oil, Apricot Oil, Jojoba Oil, Argan Oil and Hemp Seed Oil to the butter and beeswax mixture. Then, add the Vitamin E to the mixture, and continue to stir gently. Last, add the fragrance to the mixture, and gently stir. Place the mixture in jars and place in a cooling device at a temperature of 40° F. or cooler. Allow the mixture to solidify and remove from the cooling device.

The most preferred embodiment comprises 20.5% Sweet Almond Oil, 10% Apricot Oil, 10% Jojoba Oil, 10% Argan Oil, 10% Hemp Seed Oil, 12% Beeswax, 12% Shea Butter, 12% Mango Butter, 1.5% Vitamin E, and 2% Fragrance.

### Example 5: Beard Moisturizer Formula

The most preferred embodiment comprises 3% Sweet Almond Oil, 3% Apricot Oil, 3% Jojoba Oil, 3% Argan Oil, 2% Hemp Seed Oil, 2.5% Shea Butter, 2.5% Mango Butter, 3% Cetyl Alcohol, 2% Beeswax, 51.5% Distilled Water, 3% Glycerin, 0.5% Xanthan Gum, 5% Oat Extract, 5% Emulsifier, 5% Bamboo Extract, 1% Vitamin E, 2% Fragrance and 3% Preservative.

Combine Sweet Almond Oil, Apricot Oil, Jojoba Oil, Argan Oil, Hemp Seed Oil, Shea Butter, Mango Butter, Cetyl Alcohol and Olivem 1000. This is the oil phase.

Mix the xanthan gum and glycerin together. Pour the xanthan gum and glycerin mixture into the distilled water. Stir gently. Add the Oat Extract and Bamboo Extract to the distilled water. Slowly stir with a spoon or lab stirrer.

Heat the oil phase to 158° F. and until everything has melted. Heat the water phase to 158° F. Pour the oil phase into the water phase and stir gently or use a lab stirrer to stir the mixture until it turns to a cream. Then add the fragrance to the mixture and continue to stir gently. Then, add the Leucidal SF Complete or another preservative, and continue to stir in the mixture.

The embodiments of the present invention as herein disclosed provide the advantages stated herein and these examples are merely illustrative of the presently preferred embodiments and that no limitations are intended to the details of the manufacture or design.

What is claimed is:

1. A topically applied skin care body butter comprised of Shea Butter, Mango Butter, Fractionated Coconut Oil, Apricot Oil, Avocado Oil, Beeswax, Arrowroot Powder, Vitamin E and Fragrance.

2. The body butter of claim 1 comprised of from about 20% to about 45% Shea Butter, about 20% to about 30% Mango Butter, about 20% to about 30% Fractionated Coconut Oil, about 5% to about 20% Apricot Oil, about 5% to about 15% Avocado Oil, from about 5% to about 15% Beeswax, from about 2% to about 10% Arrowroot Powder, from about 1% to about 2% Vitamin E and from about 1% to about 3% Fragrance.

3. The body butter of claim 2 comprised of 30% Shea Butter, 22% Mango Butter, 22% Fractionated Coconut Oil, 10% Apricot Oil, 5% Avocado Oil, 5% Beeswax, 4% Arrowroot Powder, 1% Vitamin E and 1% Fragrance.

4. The fragrance of any of claims 1-3 may be selected from the group consisting of lavender, sandalwood and vanilla or coconut milk, milky sandalwood and hawthorne or a combination thereof.

5. A body oil of the present invention composed of Apricot Oil, Fractionated Coconut Oil, Avocado Oil, Vitamin E, and Fragrance.

6. The body oil of claim 5 wherein the Apricot Oil ranges about from about 30% to about 50% by weight, the Fractionated Coconut Oil ranges from about 20% to about 40% by weight, the Avocado Oil ranges from about 20% to about 30% by weight, the Vitamin E ranges from about 1% to about 2% by weight, and the Fragrance ranges from about 1% to about 3% by weight.

7. The body oil of claim 6 comprising 45% Apricot Oil, 30% Fractionated Coconut Oil, 22.5% Avocado Oil, 1.5% Vitamin E, and 1% Fragrance.

8. A topically applied skin and beard care oil comprised of Sweet Almond Oil, Apricot Oil, Jojoba Oil, Argan Oil, Hemp Seed Oil, Vitamin E and Fragrance.

9. The beard oil of claim 8 comprising from 20% to 35% Sweet Almond Oil, 20% to 35% Apricot Oil, 20% to 35% Jojoba Oil, 20% to 35% Argan Oil, 5% to 15% Hemp Seed Oil, 1% to 2% Vitamin E and 1% to 3% Fragrance.

10. The beard oil of claim 9 comprised of 26.5% Sweet Almond Oil, 20% Apricot Oil, 20% Jojoba Oil, 20% Argan Oil, 10% Hemp Seed Oil, 1.5% Vitamin E and 2% Fragrance.

11. A beard balm of the present invention comprised of Sweet Almond Oil, Apricot Oil, Jojoba Oil, Argan Oil, Hemp Seed Oil, Beeswax, Shea Butter, Mango Butter, Vitamin E and Fragrance.

12. The beard balm of claim 11, comprised of from about 20% to about 35% Sweet Almond Oil, from about 10% to about 20% Apricot Oil, from about 5% to about 15% Jojoba Oil, from about 5% to about 15% Hemp Seed Oil, from about 5% to about 20% Beeswax, from about 5% to about 20% Shea Butter, from about 5% to about 20% Mango Butter, from about 1% to about 2% Vitamin E and from about 1% to about 3% Fragrance.

13. The beard balm of claim 12 comprised of 20.5% Sweet Almond Oil, 10% Apricot Oil, 10% Jojoba Oil, 10% Argan Oil, 10% Hemp Seed Oil, 12% Beeswax, 12% Shea Butter, 12% Mango Butter, 1.5% Vitamin E and 2% Fragrance.

14. A topically applied skin and beard care beard moisturizing product comprised of Sweet Almond Oil, Apricot Oil, Jojoba Oil, Argan Oil, Hemp Seed Oil, Shea Butter, Mango Butter, Emulsifier, Cetyl Alcohol, Beeswax, Distilled Water, Glycerin, Xanthan Gum, Oat Extract, and Bamboo Extract. Vitamin E, Fragrance, and Preservative.

15. The beard moisturizing formula of claim 14 comprising from about 2% to about 10% Sweet Almond Oil, from about 2% to about 10% Apricot Oil, from about 2% to about 10% Jojoba Oil, from about 2% to about 10% Argan Oil, from about 1% to about 5% Hemp Seed Oil, from about 1% to about 5% Shea Butter, from about 1% to about 5% Mango Butter, from about 1% to about 10% Emulsifier, from about 2% to about 10% Beeswax, from about 45% to about 60% Distilled Water, from about 2% to about 10% Glycerin, from about 0.2% to about 3% Xanthan Gum, from about 5% to about 15% Oat Extract, from about 5% to about 15% Bamboo Extract, from about 1% to about 2% Vitamin E, from about 1% to about 3% Fragrance, and from about 1% to about 5% Preservative.

16. The skin and beard care beard moisturizing product of claim 15 comprised of 3% Sweet Almond Oil, 3% Apricot Oil, 3% Jojoba Oil, 3% Argan Oil, 2% Hemp Seed Oil, 2.5% Shea Butter, 2.5% Mango Butter, 5% Emulsifier, 3% Cetyl Alcohol, 2% Beeswax, 51.5% Distilled Water, 3% Glycerin, 0.5% Xanthan Gum, 5% Oat Extract, 5% Bamboo Extract, 1% Vitamin E, 2% Fragrance and 3% Preservative.
